(19) United States
(12) Patent Application Publication
(10) Pub. No.: US 2018/0140519 A1
(43) Pub. Date: May 24, 2018

(54) ASSEMBLY AND PROCESS FOR MAKING UP A KERATIN MATERIAL

(71) Applicant: L'OREAL, Paris (FR)

(72) Inventors: Jean-Dominique BAZIN DE BEZONS, Montrouge (FR); Dolores COLOMBEL, L'Hay-Les-Roses (FR)

(73) Assignee: L'OREAL, Paris (FR)

(21) Appl. No.: 15/316,214

(22) PCT Filed: May 22, 2015
(86) PCT No.: PCT/EP2015/061417

§ 371 Date: Dec. 5, 2016

(30) Foreign Application Priority Data
Jun. 20, 2014 (FR) 1455689

(51) Int. Cl. A61K 8/26; A61Q 3/02; A61Q 1/04; A61Q 1/10; A61Q 1/02; A61K 8/37; A61K 8/73; A61K 8/49; A61K 8/20; A61K 8/29; A61K 8/46; A61K 8/19; A61K 8/58; A61K 8/31; A61K 8/898; A61K 8/891; A61K 8/92; A61K 8/81

(52) U.S. Cl. CPC A61K 8/26; A61K 2800/438; A61Q 1/04; A61Q 1/10; A61Q 1/02; A61K 8/37; A61K 8/731; A61K 8/4973; A61K 8/20; A61K 8/29; A61K 8/466; A61K 8/498; A61K 8/19; A61K 8/585; A61K 8/31; A61K 8/898; A61K 8/891; A61K 8/927; A61K 8/92; A61K 8/8152; A61K 2800/884; A61K 2800/26; A61K 2800/262; A61K 2800/43; A61K 2800/60; A61K 2800/434; A61K 2800/436; A61Q 3/02

(57) ABSTRACT

The present invention relates to an assembly for making up a keratin material, in particular the nails, the lips, the eyelids, the eyelashes, the eyebrows or the contour of the eyes, comprising at least: —a first composition intended to be applied to the keratin material, the first composition being a composition that is capable of forming a reflective coat, —a second composition intended to be applied onto the first composition, said second composition being a translucent and coloured composition. The present invention also relates to a makeup process.

The present invention relates to an assembly for making up a keratin material, in particular the nails, the lips, the eyelids, the eyelashes, the eyebrows or the contour of the eyes, comprising a first particular composition applied to the keratin material and a second particular composition, different from the first composition, applied successively onto said first composition. The present invention also relates to a process for making up such a keratin material.

Consumers are seeking, in makeup products, radically innovative products that are capable of surprising them.

To satisfy their need, the cosmetic industries launch makeup products with broadened colour ranges, or combine these products with application devices, some of which are intended to create novel effects or novel textures.

However, these makeup products may have a high cost, may have optical or sensory effects that do not find unanimous approval among consumers, or alternatively may create these effects at the expense of the cosmetic properties usually sought in makeup products such as the staying power, the comfort or the amount of product deposited.

Makeup products are generally applied by the user applying several coats so as to arrive at an amount of product deposited judged as being satisfactory. In certain makeup applications, certain makeup products recommend the application of a first composition to the keratin material, more commonly known as the base coat, and the successive application of a second composition onto said first composition, more commonly known as the top coat.

The base coat may have several functions.

This base coat may be a coat of coloured composition protected with a transparent and colourless top coat. In such a case, the top coat is generally intended to protect said base coat from degrading over time (scratching, flaking or chipping) or is intended to improve its staying power over time. Such a top coat thus forms a shield for the base coat with regard to the users' everyday conditions, in particular water, detergent products, soaps or climatic conditions that are liable to affect the integrity of the makeup products applied to the keratin materials.

This base coat may also form a transparent and colourless substrate intended to facilitate the attachment of an opaque and coloured top coat, especially to smooth keratin materials, such as the nails. Moreover, such a configuration can, here also, give rise to the application of a second transparent and colourless top coat intended to protect the opaque and coloured top coat.

One aim of the invention is thus to propose an assembly and a process for making up keratin materials that are capable of creating surprising optical effects, in particular a depth effect.

One aim of the invention is thus to propose an assembly and a process for making up keratin materials that are capable of creating optical effects that are available in several makeup uses, such as for making up the nails, the lips, and skin such as the eyelids or the contour of the eyes.

One aim of the invention is also to propose a customizable assembly and process for making up keratin materials, offering a wide choice of colours.

One aim of the invention is also to propose an applicable assembly and process for making up keratin materials that make it possible to adapt existing formulations, that are readily industrializable, and readily usable, with an easily reproducible optical effect.

One subject of the present invention is thus, according to a first aspect that satisfies at least one of the abovementioned problems, an assembly for making up a keratin material, in particular the nails, the lips, the eyelids, the eyelashes, the eyebrows or the contour of the eyes, comprising at least:

a first composition intended to be applied to the keratin material, the first composition being a composition that is capable of forming a reflective coat, and
a second composition intended to be applied onto the first composition, said second composition being a translucent and coloured composition.

After application of the first composition in the form of a base coat in contact with the keratin material, for example the nails, and then of the second composition in the form of a coat covering the base coat, the user may observe that, by looking at the deposit made, the covering coat makes it possible to create a depth effect of said base coat.

With the present invention, it is possible to apply a plurality of covering coats for darkening the shade while at the same time being very saturated (intense colour), making it possible to obtain a “deep” shade (possibility of dark coloration while at the same time remaining translucent, allowing the base coat to show through). Furthermore, the present invention makes it possible to modify the lightness and saturation of the shade obtained with the second composition applied by varying the number of coats deposited. A depth effect is obtained, thus resulting from high chromaticity (strong shade) combined, on the one hand, with a reduction in lightness (as a function of the number of covering coats deposited), and, on the other hand, with a certain translucency of this (these) covering coats, to perceive the light reflected by the base coat. The translucency of the covering coat allows light to be reflected onto the base coat, making it possible to amplify the chromaticity of the covering coat (intensity of the colour).

In the patent application EP1745770, we know an assembly for make-up comprising a first composition, containing a resin selected from colophane and derivatives thereof, hydrocarbon-based resins and their mixtures, the said resin having a molecular weight in number inferior or equal to 10000 g/mol and a second composition containing at least one non volatile fatty substance. Example 1 describes an assembly for making-up of the lips comprising a red lip make-up composition and a non colored second gloss composition.

In the patent application FR2932981, we also know a make-up assembly of two compositions whererin the first composition comprises a colouring agent and the second composition comprises a non polymerized or non polymerisable liquid crystal. The patent application FR2908637 describes a make-up assembly of two compositions wherein the first composition contains a multilayer interference pigment and presents a coverage of at least 30, the amount of multilayer interference pigment leading the composition to a colour change ΔE of at least 2 between the colour in the mass and the colour after application.

No one of those make-up assemblies allows to obtain those optical and coloring properties.

According to particular embodiments satisfying at least one of the abovementioned technical problems, the makeup assembly according to the invention has the following technical characteristics:
the first composition comprises at least one reflective metal particle;
the reflective metal particle(s) are chosen from the group consisting of pure or composite metal particles, optionally oxidized, multilayer interference pigments, optionally covered with optical brighteners, and a mixture thereof, the multilayer interference pigment(s) in particular being chosen from the group consisting of nacres, goniochromatic pigments, optionally covered with optical brighteners, and a mixture thereof;
the reflective metal particle(s) are present in a total content ranging from 2% to 40% by weight, more precisely from 3% to 30% by weight and preferably from 4% to 20% by weight, relative to the total weight of the first composition;
when the first composition is a nail varnish, the reflective metal particle(s) are present in a total content ranging from 2% to 20% by weight and more precisely from 4% to 15% by weight relative to the total weight of the first composition;
when the first composition is a lipstick, the reflective metal particle(s) are present in a total content ranging from 2% to 40% by weight and more precisely from 4% to 30% by weight, relative to the total weight of the first composition;
the first composition has a colour result chosen from the group of colours consisting of white, pink, metallic, in particular silvery or golden, and pastel;
the first composition has a contrast ratio CR of greater than or equal to 75%, better still 80%, more advantageously 85%, in particular from 75% to 100%, a chromaticity C* of less than or equal to 40, preferably 30, better still 20, more advantageously 10, in particular 5 or even 2.5, and preferably a luminance L* of greater than or equal to 60, preferentially from 65 to 100, in accordance with the measuring protocols defined in the description;
the first composition comprises at least one volatile solvent, in particular an organic solvent, the volatile solvent(s) preferably being present in a total content ranging from 20% to 80% by weight, especially from 30% to 75% by weight and in particular from 40% to 70% by weight, relative to the total weight of the first composition;
the first composition comprises at least one film-forming polymer, the film-forming polymer(s) being present in a total content ranging from 1% to 50% by weight, better still from 5% to 40% by weight and in particular from 10% to 25% by weight, relative to the total weight of the first composition;
the first composition comprises at least one plasticizer, the plasticizer(s) preferably being present in a total content ranging from 1% to 15% and preferably from 1% to 7% by weight, relative to the total weight of the first composition;
the first composition comprises reflective metal particles that have undergone a milling step during the process for manufacturing said first composition;
the second composition comprises at least one dyestuff chosen from the group consisting of organic pigments, coloured fluorescent agents and a mixture thereof, preferably from the group consisting of organic pigments;
the second composition comprises at least one dyestuff, the dyestuff(s) being present in a total content ranging from 0.05% to 5% by weight, preferably from 0.10% to 2.5% by weight, relative to the total weight of the second composition;
when the second composition is a nail varnish, the dyestuff(s) are present in a total content ranging from 0.05% to 1% by weight and preferably from 0.10% to 0.75% by weight relative to the total weight of the second composition;
when the second composition is a lipstick, the dyestuff(s) are present in a total content ranging from 0.5% to 5% by weight and preferably from 1% to 4% by weight relative to the total weight of the second composition;
the second composition has a colour result chosen from the group consisting of coloured organic pigments, or lakes, certain coloured mineral pigments (such as CI77510, CI77007), coloured hybrid pigments, coloured fluorescent agents, and a mixture thereof, preferably from coloured organic pigments, coloured fluorescent agents, and a mixture thereof, preferably from coloured organic pigments;
the second composition has a contrast ratio CR ranging from 15% to 70%, in particular from 20% to 50%, a chromaticity C* ranging from 30 to 90 and preferably from 35 to 75, and preferably a luminance L* of greater than or equal to 40, in particular ranging from 45 to 90, in accordance with the measuring protocols defined hereinafter in the description;
the second composition comprises at least one volatile solvent, in particular an organic solvent, the volatile solvent(s) preferably being present in a total content ranging from 20% to 80% by weight, especially from 30% to 75% by weight and in particular from 40% to 70% by weight, relative to the total weight of the second composition;
the second composition comprises at least one film-forming polymer, the film-forming polymer(s) being present in a total content ranging from 1% to 50% by weight, better still from 5% to 40% by weight and in particular from 10% to 25% by weight, relative to the total weight of the second composition;
the second composition comprises at least one plasticizer, the plasticizer(s) preferably being present in a total content ranging from 1% to 15% and preferably from 1% to 7% by weight relative to the total weight of the second composition;
the first composition comprises dyestuffs that have undergone a milling step during the process for manufacturing said second composition.
   The protocols for measuring the parameters mentioned above are as follows.

Measurement of the Contrast Ratio (CR)

The application may be performed with an automatic spreader.

The contrast ratio of the first and second compositions is evaluated by preparing a 300 μm film on a contrast card (such as a Byk Chart PA-2814) having a white part and a black part, using an automatic spreader.

The film is left to dry for 24 hours on a thermostatic bed at 30° C. and a measurement on the white background is then taken.

Colour measurements were taken using a Datacolor 600 spectrocolorimeter. The colorimeter gives numerical data representing the absolute value and the colour difference between a reference sample and a sample to be controlled. In our study, we use the absolute values of each film sample on the white background for the colour, integrating the black background for the calculation of the contrast ratio.

The operating conditions are as follows:
CIE 1964 10° standard observer
illuminant CIE type D65
measuring geometry: scattered measuring geometry/8° and observation, D65/10°, specular component included (SCI) mode, mean aperture (20 mm) on the white and black backgrounds.

The apparatus is equipped with an optical device that produces scattered light, placed in a spherical cavity lined with a white coat, which induces multiple reflection of the light. An anti-glare trap can remove the glare effects from the surface of the sample.

The spectra are expressed as colorimetric coordinates in the Commission Internationale de l'Eclairage CIELab76 space according to recommendation 15:2004.

The contrast ratio is calculated by taking the arithmetic mean of the Y values on the black background, divided by the mean value of Y on the white background, multiplied by 100.

$$Contrast Ratio = \frac{Mean ( Yblack )}{Mean ( Ywhite )} * 100$$

Luminance: L*

The luminance obtained with said first and second compositions is evaluated visually on the white background of a Byk Chart PA-2814 contrast card and read using a Datacolor 600 spectrocolorimeter (illuminant D65, angle 10°, SCI values) for the L* colorimetric measurements. In an L* a* b* system, the lower the value of L*, the darker the colour.
Chromaticity: C*

The chromaticity of the first and second compositions in the CIE L*, a*, b* system is calculated according to the following equation:

C*=\sqrt{a*²+b*²}

The higher the value of C*, the more saturated the coloration obtained.

A subject of the present invention is also, according to a second aspect satisfying at least one of the abovementioned problems, a process for making up a keratin material, in particular the nails, the lips, the eyelids, the eyelashes, the eyebrows or the contour of the eyes, using a makeup assembly as defined previously, comprising, preferably consisting of:
at least a first step of applying a first composition to said keratin material, said first composition forming a reflective coat, and
at least a second step, successive to said first step, of applying a second composition onto said first composition, said second composition being a translucent and coloured composition.

According to particular embodiments satisfying at least one of the abovementioned technical problems, the makeup process according to the invention involves the following steps:
at least one drying step, preferably in the open air, of said first composition applied, before applying said second composition.

Needless to say, the particular characteristics defining the first and second compositions are also valid for this makeup process.

Makeup Assembly

A makeup assembly according to the invention comprises:
a first composition intended to be applied to the keratin material, the first composition being a composition that is capable of forming a reflective coat, and
a second composition intended to be applied onto the first composition, said second composition being a translucent and coloured composition.

Such an assembly is intended for making up a keratin material, in particular the nails, the lips, skin such as the eyelids or the contour of the eyes, or the eyelashes or the eyebrows, preferably the nails, the lips and the skin, more preferentially the nails.

The invention may thus also be in the form of a packaging and makeup device comprising:

i) a first container comprising an applicator suitable for applying said first composition to the keratin material,

ii) a second container comprising an applicator suitable for applying said first composition to the keratin material.

This first composition and this second composition may be packaged in a unit or non-unit device.

The unit device may be, for example, in the form of a tube. This unit device may comprise, for example, the first and second containers integrally attached via suitable fixing means by means of a connecting piece.

When the device is a non-unit device, the containers are in separate form and may be sold, for example, by being contained in the same packing or separately with explicit recommendations for the respective use of the first and second containers.

First Composition

The first composition is preferably intended to be applied directly to the keratin material, in direct contact with the keratin material.

The keratin material is preferably chosen from the group consisting of the nails, the lips, skin such as the eyelids or the contour of the eyes, the eyelashes and the eyebrows, preferably the nails, the lips and the skin, more preferentially the nails.

This first composition is preferably intended to form a base coat for the second composition.

According to the invention, this first composition is capable of forming a reflective coat after application to said keratin material.

The term “reflective coat” means that the first composition has:
a contrast ratio of greater than or equal to 75%, better still 80%, more advantageously 85%, in particular ranging from 75% to 100%,
a chromaticity C* of less than or equal to 40, in particular 30, better still 20, more advantageously 10, preferably 5, or even 2.5,
preferably, a luminance, or reflectance, L* of greater than or equal to 60, in particular from 65 to 100, in accordance with the protocols given earlier in the description. It should be noted that these values are obtained on a coat of first composition alone.

Preferably, a first composition according to the invention has a white, pink, metallic, in particular silvery or golden, or pastel colour result.

This first composition comprises at least one reflective metal particle.

Reflective Metal Particles

The term “reflective particles” denotes particles whose size, structure, especially the thickness of the layer(s) of which they are constituted and their physical and chemical natures, and the surface appearance, enable them to reflect incident light with a sufficient intensity to be able to create at the surface of the composition claimed, when the latter is applied to the support to be made up, points of brightness that are visible to the naked eye, i.e. more luminous points that contrast with their surroundings by appearing to shine.

The reflective particles may disrupt the visual perception of the curvature of the made-up support, by tending to prevent lasting visual focusing, the points of brightness being liable to appear or disappear at random when the made-up support and the observer are moving.

The reflective particles used must be compatible with cosmetic use and must be able to remain in the composition, and especially to not dissolve, or in any case to not dissolve entirely, therein.

The reflective particles advantageously have a size that is compatible with the manifestation of specular reflection of visible light (400-700 nm), of sufficient intensity to create a point of brightness. This size is liable to vary according to the chemical nature of the particles, their shape and their power for specular reflection of visible light.

The term “metal” means comprising, or consisting of, at least one metal, a metal alloy or a metal (poly)oxide.

The reflective metal particles present in this first composition are preferably chosen from:
pure isolated metal particles, optionally oxidized, in particular chosen from aluminium, bronze, silver, gold, bismuth oxychloride and titanium dioxide, and a mixture thereof;
composite metal particles, which may in particular result from an alloy of several metals, at least one of the metals being optionally oxidized,
metal particles in layer form, especially of the type such as multilayer interference pigments, in particular covering a natural or synthetic substrate, preferably a natural or synthetic mineral substrate, optionally composites on a substrate, optionally multilayer, the metal(s) optionally being oxidized;
and a mixture thereof.

A first composition according to the invention advantageously comprises a total content of reflective metal particles ranging from 2% to 40% by weight, more precisely from 3% to 30% by weight and preferably from 4% to 20% by weight, relative to the total weight of the first composition.

A/ Pure or Composite, Optionally Oxidized Metal Particles

A first composition according to the invention may comprise, as reflective metal particles, pure or composite, optionally oxidized metal particles.

Such particles may be chosen, for example, from aluminium alone or in composite form (Cosmicolor®), bismuth oxychloride, bronze, silver, gold and titanium oxide, and a mixture thereof.

A first composition according to the invention may comprise a total content of such particles ranging from 2% to 40% by weight, more precisely from 3% to 30% by weight and preferably from 4% to 20% by weight, relative to the total weight of the first composition.

B/ Multilayer Interference Pigments

A first composition according to the invention may comprise, as reflective metal particles, one or more multilayer interference pigments.

The term “multilayer interference pigment” denotes a pigment that is capable of producing a colour via an interference phenomenon between light rays reflected by a plurality of superposed layers of different refractive indices, especially a succession of layers of high and low refractive indices.

All multilayer interference pigments may be envisaged.

The colour produced by the multilayer interference pigment may be any optionally having, for example, a dominant wavelength of between 580 and 650 nm.

The composition may comprise a single multilayer interference pigment or several multilayer interference pigments having different dominant wavelengths.

The multilayer interference pigment may comprise a substrate (also known as a core or nucleus) covered on at least one face with one or more layers of materials and thicknesses chosen such that a colour is produced by interference.

Layers of the interference pigment may or may not surround the substrate, which may or may not have a flattened shape.

The substrate may comprise natural mica, synthetic mica, glass, alumina, silica, or any metal, alloy or metal oxide. The nature of the substrate may be chosen as a function of the desired gloss. For example, for a very glossy result, a glass or metal substrate may be preferred.

The interference pigment may comprise more than four layers of different refractive indices.

The size of the multilayer interference pigment particles, given by the mean granulometry at half the population, also known as D₅₀, ranges, for example, from 1 μm to 2000 μm and better still from 5 μm to 2000 μm.

The contrast ratio of the composition may be essentially due to the content of multilayer interference pigment.

The multilayer interference pigments may be chosen from nacres and goniochromatic pigments, and a mixture thereof.

A first composition according to the invention advantageously comprises a total content of multilayer interference pigments ranging from 2% to 40% by weight, more precisely from 3% to 30% by weight and preferably from 4% to 20% by weight, relative to the total weight of the first composition.

a) Nacres

A first composition according to the invention may comprise one or more nacres.

The term “nacre” should be understood as meaning coloured particles of any form, which may or may not be iridescent, especially produced by certain molluses in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica coated especially with an organic dye, and also nacreous pigments based on bismuth oxychloride.

They may also be mica particles, at the surface of which are superposed at least two successive layers of metal oxides and/or of organic dyestuffs.

These particles may be chosen from particles containing a natural or synthetic substrate at least partially coated with at least one layer of at least one metal oxide, chosen, for example, from titanium oxides, especially TiO₂, iron oxides, especially Fe₂O₃, tin oxides, chromium oxides, barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

Examples of such particles that may be mentioned include particles comprising a synthetic mica substrate coated with titanium dioxide, or glass particles coated either with brown iron oxide, with titanium oxide, with tin oxide or with a mixture thereof, for instance the products sold under the brand name Reflecks® by the company Engelhard.

The nacres may have a yellow, pink, red, bronze, orangey, brown, gold and/or coppery tint.

Non-limiting examples of nacres that may be mentioned in particular, alone or as a mixture, include nacres comprising the following elements:
mica-titanium oxide (for example sold under the commercial reference Timica Sparkle 110P from BASF, Flamenco Blue from BASF, Candurin Brown Amber from Merck, Pearlescent Pigment Prestige Gold from Sudarshan, etc.);
mica-titanium oxide coated with N-lauroyl-L-lysine (for example sold under the commercial reference LLD-10 Flamenco Violet from Daito Kasei Kogyo, etc.);
mica-iron oxide (for example sold under the commercial reference Pearlescent Pigment Prestige Soft Bronze from Sudarshan, Colorona Bronze Sparkle from Merck, Timica Golden Bronze from BASF, Candurin Light Gold from Merck, Duocrome YR 422 C from BASF, Gemtone Tan Opal G 005 from BASF, etc.);
mica-titanium oxide-iron oxide (for example sold under the commercial reference Timica Brilliant Gold 212 G Gemtone Goldstone G0014 or Timica Golden Bronze from BASF, etc.);
mica-bismuth oxychloride-iron oxide (for example sold under the commercial reference Chroma-Lite Yellow from BASF, etc.);
mica-titanium oxide-chromium oxide-iron oxide (for example sold under the commercial reference Gemtone Jade from BASF, etc.);
mica-titanium oxide-tin oxide (for example sold under the commercial reference Flamenco Super Blue 630 Z from BASF, Helios R100R from Topy, etc.);
mica-silica-titanium oxide (for example sold under the commercial reference Timiron Splendid Red from Merck, etc.);
platelets of sodium calcium borosilicate coated with titanium oxide and tin oxide (for example sold under the commercial reference Reflecks Rays of Red G430L from BASF, etc.);
platelets of sodium calcium borosilicate coated with tin oxide and iron oxide (for example sold under the commercial reference Mirage Sparkling Champagne from Eckart, etc.);
platelets of calcium aluminium borosilicate coated with titanium oxide and tin oxide, optionally treated with silica (for example sold under the commercial reference Ronastar from Merck, etc.);
platelets of calcium aluminium borosilicate coated with silicon oxide and tin oxide (for example sold under the commercial reference Ronastar Golden Jewel SQ from Merck, etc.);
glass powder coated with titanium oxide (for example sold under the commercial reference Reflecks Dimensions Glittering Gold G230S from BASF or Metashine MC1120RY from NSG, etc.);
alumina and titanium oxide (for example sold under the commercial reference Spectraflex Focus Red C88-1031 from Sun, etc.);
   it being understood that the mica may be natural or synthetic and it being understood that oxide is equivalent to (poly)oxide.

Thus, according to one advantageous embodiment, the reflective metal particles in accordance with the invention comprise at least one element chosen from the following CI (Color Index) numbers: 77000, 77002, 77004, 77019, 77120, 77163, 77220, 77231, 77400, 77480, 77711, 77718, 77820, 77891, 77947, and a mixture thereof, it being understood that the reflective metal particles comprise at least one metal element optionally in (poly)oxide form.

Preferably, a composition in accordance with the invention comprises nacres of white or silvery colour.

A first composition according to the invention advantageously comprises a total content of nacre(s) ranging from 2% to 40% by weight, more precisely from 3% to 30% by weight and preferably from 4% to 20% by weight, relative to the total weight of the first composition.

b) Goniochromatic pigments

A first composition according to the invention may comprise one or more goniochromatic pigments.

For the purposes of the present invention, the term “goniochromatic pigment” denotes a pigment which makes it possible to obtain, when the composition is spread onto a support, a colour trajectory in the a*b* plane of the CIE 1976 colorimetric space that corresponds to a variation Dh ° in the hue angle h ° of at least 20° C. when the angle of observation relative to the normal is varied between 0° and 80°, for an incident light angle of 45°.

The colour trajectory may be measured, for example, using an Instrument Systems brand spectrogonioreflectometer of reference GON 360 Goniometer, after the composition has been spread in fluid form to a thickness of 300 μm using an automatic spreader onto an Erichsen brand contrast card of reference Typ 24/5, the measurement being taken on the black background of the card.

The goniochromatic pigment may be chosen, for example, from multilayer interference structures and liquid-crystal colouring agents.

In the case of a multilayer structure, it may comprise, for example, at least two layers, each layer being made, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, and alloys, polymers and combinations thereof.

The multilayer structure may or may not have, relative to a central layer, symmetry in the chemical nature of the stacked layers.

Different effects are obtained depending on the thickness and the nature of the various layers.

Examples of symmetrical multilayer interference structures are, for example, the following structures: Fe₂O₃/SiO₂/Fe₂O₃/SiO₂/Fe₂O₃, a pigment having this structure being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the name Xirona by the company Merck (Darmstadt).

The liquid-crystal colouring agents comprise, for example, silicones or cellulose ethers onto which are grafted mesomorphic groups. Examples of liquid-crystal goniochromatic particles that may be used include, for example, those sold by the company Chenix and also the products sold under the name Helicone® HC by the company Wacker.

Goniochromatic pigments that may also be used include certain nacres, pigments with effects on a synthetic substrate, especially a substrate such as alumina, silica, borosilicate, iron oxide or aluminium, or interference flakes obtained from a polyterephthalate film.

Non-limiting examples of goniochromatic pigments that may be mentioned in particular, alone or as a mixture, include the following goniochromatic pigments:
synthetic mica-titanium oxide-silica-tin oxide (for example sold under the commercial reference Sunshine by Sun, etc.);
platelets of aluminium covered with silica as interference layer and silver nanoparticles as outer layer (for example sold under the commercial reference Cosmicolor Celeste by Toyo Aluminium K.K., etc.);
silica-titanium oxide-(mica-)tin oxide (for example sold under the commercial reference Xirona by Merck);
platelets of calcium and sodium-silica-titanium oxide-tin oxide (for example sold under the commercial reference Reflecks Multidimensions by BASF).

A first composition according to the invention advantageously comprises a total content of goniochromatic pigments ranging from 2% to 40% by weight, more precisely from 3% to 30% by weight and preferably from 4% to 20% by weight, relative to the total weight of the first composition.

These reflective metal particles and in particular these multilayer interference pigments may optionally comprise or be covered with optical brighteners.

Brighteners are white organic fluorescent substances.

Optical brighteners are compounds well known to a person skilled in the art. Such compounds are described in “Fluorescent Whitening Agent, Encyclopedia of Chemical Technology, Kirk-Othmer”, vol. 11, pp. 227-241, 4th Edition, 1994, Wiley. Their use in cosmetics in particular exploits the fact that they consist of chemical compounds having fluorescence properties, which absorb in the ultraviolet region (maximum absorption at a wavelength of less than 400 nm) and re-emit energy by fluorescence for a wavelength of between 380 nm and 830 nm.

They may be defined more particularly as compounds that absorb essentially in the UVA region between 300 and 390 nm and re-emit essentially between 400 and 525 nm.

Their lightening effect resides more particularly in an emission of energy of between 400 and 480 nm, which corresponds to an emission in the blue part of the visible region, which contributes to lightening the skin visually when this emission takes place on the skin.

Optical brighteners that are especially known include stilbene derivatives, in particular polystyrylstilbenes and triazinylstilbenes, coumarin derivatives, in particular hydroxycoumarins and aminocoumarins, oxazole, benzoxazole, imidazole, triazole and pyrazoline derivatives, pyrene derivatives, porphyrin derivatives and mixtures thereof.

They include in particular the following derivatives:
the stilbene derivative of naphthotriazole, sold under the trade name Tinopal GS, the disodium 4,4′-distyrylbiphenylsulfonate (CTFA name: disodium distyrylbiphenyl disulfonate) sold under the trade name Tinopal CBS-X, the cationic aminocoumarin derivative sold under the trade name Tinopal SWN CONC., the sodium 4,4′-bis[(4,6 dianilino-1,3,5-triazin-2-yl)amino]stilbene-2,2′-disulfonate sold under the trade name Tinopal SOP, the 4,4′-bis[(4-anilino-6-bis(2-hydroxyethyl)amino-1,3,5-triazin-2-yl)amino]stilbene-2,2′-disulfonic acid sold under the trade name Tinopal UNPA-GX, the 4,4′-bis[(anilino-6-morpholine-1,3,5 -triazin-2-yl)amino]stilbene sold under the trade name Tinopal AMS-GX, and the disodium 4,4′-bis[(4-anilino-6-(2-hydroxyethyl)methylamino-1,3,5-triazin-2-yl)amino]stilbene-2,2′-sulfonate sold under the trade name Tinopal 5BM-GX, all by the company CIBA Specialty Chemicals;
the 2,5-thiophenediylbis(5-tent-butyl-1,3-benzoxazole) sold under the trade name Uvitex OB by the company CIBA;
the anionic derivative of diaminostilbene in dispersion in water, sold under the trade name Leucophor BSB liquide by the company Clariant; and
mixtures thereof.

The optical brighteners that can be used in the present invention may also be in the form of copolymers, for example of acrylates and/or methacrylates, grafted with optical brightener groups as described in application FR 99/10942.

These reflective metal particles may be formulated in the first composition with:
at least one volatile solvent, in particular an organic solvent,
preferably at least one film-forming polymer,
preferably at least one plasticizer,
optionally, at least one dispersant, and also any other compound that is capable of preserving the optical characteristics defined previously of the reflective coat for this first composition.

Volatile Solvents

The first composition according to the invention may comprise at least one volatile solvent, which is preferably organic.

For the purposes of the invention, the term “volatile solvent” means a solvent that is capable of evaporating on contact with keratin materials in less than one hour, at room temperature and atmospheric pressure. The volatile solvent(s) of the invention are solvents that are liquid at room temperature and that have a non-zero vapour pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile solvent of the composition is preferably chosen from the group consisting of C₂-C₅ alcohols, C₅-C₁₂ alkanes, and C₃-C₆ esters and ketones, and mixtures thereof.

The term “C₂-C₅ alcohol” means an organic compound comprising at least one hydroxyl group —OH, preferably exactly one hydroxyl group —OH, and from 2 to 5 carbon atoms, and which is moreover liquid at room temperature, i.e. from 15° C. to 30° C. The C₂-C₅ alcohols that are suited to the invention especially include ethanol and isopropanol.

The term “C₅-C₁₂ alkane” means a saturated, linear or branched, cyclic or acyclic hydrocarbon-based compound, consisting exclusively of carbon and hydrogen atoms, which is moreover liquid at room temperature, i.e. from 15° C. to 30° C. In the context of the present invention, the alkanes preferably comprise from 5 to 12, preferentially from 5 to 10 and advantageously from 5 to 8 carbon atoms. The C₅-C₁₂ alkanes that are suited to the invention especially include heptane and isomers thereof, decane and isomers thereof, cyclohexane and dodecane and isomers thereof.

C₃-C₆ esters and ketones that may especially be mentioned include acetone, methyl ethyl ketone, methyl isobutyl ketone, cyclohexanone and alkyl acetates in which the alkyl group comprises from 2 to 5 carbon atoms, such as methyl acetate, ethyl acetate, propyl acetate, n-propyl acetate, isopropyl acetate, n-butyl acetate, isobutyl acetate and tert-butyl acetate.

According to a particular embodiment, the first composition comprises a total content of volatile solvent(s) of between 20% and 80% by weight, especially between 30% and 75% by weight and in particular between 40% and 70% by weight, relative to the total weight of the first composition.

Film-Forming Polymers

The first composition according to the invention may comprise at least one film-forming polymer, which is preferably soluble in the volatile solvent(s).

For the purposes of the present invention, the term “film-forming polymer” denotes a polymer that is capable, by itself or in the presence of an auxiliary film-forming agent, of forming an isolable and especially continuous and adherent film, on a support, especially on the nails.

A single film-forming polymer or a mixture of film-forming polymers may be used in the composition.

This film-forming polymer may be chosen from the group made up of synthetic polymers, of radical type or of polycondensate type, and polymers of natural origin, and mixtures thereof.

A film-forming polymer that is suitable for use in the invention may be chosen in particular from:
cellulose-based polymers or polysaccharide derivatives, such as cellulose derivatives or guar gum derivatives. A polysaccharide derivative that is suitable for use in the invention may be a polysaccharide ester or alkyl ether.

The term “polysaccharide ester or alkyl ether” denotes a polysaccharide made up of repeat units comprising at least two identical or different rings and having a degree of substitution per saccharide unit of between 1.9 and 3, preferably between 2.2 and 2.9 and more particularly between 2.4 and 2.8. The term “substitution” denotes the functionalization of the hydroxyl groups to give ester and/or alkyl ether functions, and/or the functionalization of the carboxylic groups to give ester functions.

In other words, it may be a polysaccharide, partially or totally substituted with ester and/or alkyl ether groups. Preferably, the hydroxyl groups may be substituted with ester and/or alkyl ether functions of C₂-C₄.

Mention may in particular be made of cellulose esters, such as cellulose acetate, cellulose acetobutyrates or cellulose acetopropionates; cellulose alkyl ethers, for instance ethylcelluloses, and ethyl guars;
synthetic polymers such as homopolymers and copolymers of a compound bearing an ethylenic unit, acrylic polymers and copolymers, polyurethanes, polyesters, polyureas, silicone polymers such as silicone resins, silicone polyamides, polymers bearing a non-silicone organic backbone grafted with monomers containing a polysiloxane, polyamide polymers and copolymers, polyisoprenes, vinyl polymers, polyvinyl butyrals, alkyd resins and ketone/aldehyde resins, resins derived from aldehyde condensation products, such as arylsulfonamide-formaldehyde resins, for instance toluenesulfonamide-formaldehyde resin, epoxy/tosylamide resins, arylsulfonamide epoxy resins or ethyl tosylamide resins;
polymers of natural origin, such as plant resins, for instance dammar resins, elemi gums, copal resins, and benzoin; gums such as shellac, sandarac gum and gum mastic.

Film-forming polymers that may especially be used include the toluenesulfonamide-formaldehyde resins Ketjenflex MS80 from the company Akzo or Santolite MHP and Santolite MS 80 from the company Faconnier or Resimpol 80 from the company Pan Americana, the alkyd resin Beckosol ODE 230-70-E from the company Dainippon, the acrylic resin Acryloid B66 from the company Röhm & Haas, the polyurethane resin Trixene PR 4127 from the company Baxenden, and the acetophenone/formaldehyde resin sold under the reference Synthetic Resin SK by Degussa.

According to a particular embodiment, the film-forming polymer is chosen from polysaccharides and polysaccharide derivatives, preferably from polysaccharide ethers and esters, especially of C₂-C₄, and is more preferentially chosen from cellulose acetobutyrates, cellulose acetopropionates, cellulose acetate, ethylcelluloses, ethyl guars, nitrocellulose, epoxy/tosylamide resins and acrylic polymers, and mixtures thereof.

According to a particular embodiment, the first composition comprises a total content of film-forming polymer(s) of between 1% and 50% by weight, especially between 5% and 40% by weight and in particular between 10% and 25% by weight, relative to the total weight of the first composition.

Plasticizers

The first composition according to the invention may comprise at least one plasticizer.

For the purposes of the present invention, the term “plasticizer” denotes a compound, in more or less viscous liquid form, which, when added to a formulation of a given material, such as a polymer, enables said material to be rendered more tacky, more adherent, more flexible, more resistant and/or easier to handle.

Plasticizers that may especially be mentioned include the usual plasticizers, such as glycols and derivatives thereof, such as diethylene glycol ethyl ether, diethylene glycol methyl ether, diethylene glycol butyl ether or else diethylene glycol hexyl ether, ethylene glycol ethyl ether, ethylene glycol methyl ether, ethylene glycol butyl ether or ethylene glycol hexyl ether; glycol esters; propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether or propylene glycol butyl ether; acid esters, especially carboxylic acid esters, such as citrates, especially triethyl citrate, tributyl citrate, triethyl acetylcitrate, tributyl acetylcitrate or 2-triethylhexyl acetylcitrate; phthalates, in particular dimethoxyethyl phthalate; phosphates, in particular tricresyl phosphate, tributyl phosphate, triphenyl phosphate or tributoxyethyl phosphate; tartrates, especially dibutyl tartrate; adipates; carbonates; sebacates; benzyl benzoate, butyl acetylricinoleate, glyceryl acetylricinoleate, butyl glyco late, camphor, glyceryl triacetate or N-ethyl-o,p-toluenesulfonamide; oxyethylenated derivatives such as oxyethylenated oils, in particular vegetable oils, such as castor oil, and more generally non-volatile oils, in particular non-volatile silicone oils, non-volatile hydrocarbon-based oils, and mixtures thereof.

Preferably, the content of plasticizer(s) is from 1% to 15% and preferably from 1% to 7% by weight, relative to the total weight of the first composition.

Thixotropic Agents

The first composition according to the invention may comprise at least one thixotropic agent.

Preferably, this (these) agent(s) are chosen from Bentone 27G (stearalkonium hectorite from Elementis) and silica (Aerosil 200 sold by Evonik Degussa).

Preferably, the content of thixotropic agent(s) is from 0.1% to 5% and preferably from 0.5% to 3% by weight, relative to the total weight of the first composition.

Needless to say, other compounds may be present in the first composition, provided that these compounds do not disrupt the reflective nature of the first composition applied.

Second Composition

The second composition is intended to be applied directly onto the first composition placed in contact beforehand with the keratin material in the form of one or more coats.

This second composition is preferably intended to form a covering coat, more commonly referred to as a top coat, intended to cover the base coat(s) applied previously.

This second composition is, according to the invention, capable of forming, after application to said keratin material, a coloured translucent coat.

The term “coloured translucent coat” means that the second composition has:
a low contrast ratio ranging from 15% to 70% and in particular from 20% to 50%,
a chromaticity C* ranging from 30 to 90 and preferably from 35 to 75,
a luminance of greater than or equal to 40, in particular ranging from 45 to 90, in accordance with the protocols given earlier in the description. It should be noted that these values are obtained on a coat of second composition alone (not applied onto a coat of first composition).

These optical values are obtained with a coat of second composition of thickness defined previously. However, it should be noted that these results are advantageously also observed after applying two coats of second composition (600 μm). Thus, preferably, even after reapplying a second coat of second composition onto the first coat, the second composition maintains its coloured translucent nature. Application of this second coat makes it possible, surprisingly, to give a depth effect to the first coat applied previously to the nail.

A second composition according to the invention has, for example, and without being limiting, a colour result of orange, fuchsia, violet, yellow, blue, green, pink, etc. colour.

The dyestuff(s) present in this second composition are preferably chosen from:
coloured organic pigments,
coloured fluorescent agents,
and a mixture thereof.

A second composition according to the invention advantageously comprises a total content of dyestuff(s) ranging from 0.12% to 0.55% (for RAL silicone: 2.45% of organic pigment) (see p.4).

Coloured Organic Pigments

A second composition according to the invention may comprise one or more coloured organic pigments.

The term “coloured organic pigments” should be understood as meaning coloured, organic particles of any shape, which are insoluble in the physiological medium, and which are intended to colour the composition.

The organic pigments may be chosen, for example, from the materials below, and mixtures thereof:
cochineal carmine,
organic pigments of azo dyes, anthraquinone dyes, indigoid dyes, xanthene dyes, pyrene dyes, quinoline dyes, triphenylmethane dyes or fluoran dyes.
   Among the organic pigments, mention may be made especially of the D&C certified pigments known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.
mineral pigments, for instance ultramarines/CI 77007 and ferric ammonium ferrocyanide.

The chemical materials corresponding to each of the organic dyestuffs mentioned previously are mentioned in the publication International Cosmetic Ingredient Dictionary and Handbook, 1997 edition, pages 371 to 386 and 524 to 528, published by The Cosmetic, Toiletries and Fragrance Association, the content of which is incorporated into the present patent application by reference.

A second composition according to the invention may comprise a total content of pigments ranging from 0.05% to 5% by weight and preferably from 0.10% to 2.5% by weight, relative to the total weight of the second composition.

Coloured Fluorescent Agents

The dyestuff(s) present in the second composition may be chosen from coloured (other than white) fluorescent agents.

Fluorescent agents are well known to those skilled in the art. They may be pigments or dyes. The term “pigments” is understood to mean inorganic or organic particles that are insoluble in the composition. The term “dye” is understood to mean chemical compounds dissolved in the composition. The dyes may be water-soluble or fat-soluble. Fluorescent substances are, for example, described in “Luminescent materials (fluorescent daylight)”, Encyclopedia of Chemical Technology, Kirk-Othmer, vol. 14, pp. 546-569, 3rd edition, 1981, Wiley.

For the purposes of the present invention, a fluorescent agent is a substance which, under the effect of ultraviolet rays and/or of visible light, re-emits in the visible range the portion of light which it has absorbed under the same colour as that which it reflects naturally. The naturally reflected colour is thus reinforced by the re-emitted colour and appears extremely bright.

As fluorescent agent, use may be made of inorganic fluorescent substances such as those described in application JP 05-117127 and, in particular, inorganic fluorescent substances based on zinc oxide.

Fluorescent agents that may also be used include organic fluorescent substances such as daylight-fluorescent pigments; these pigments are generally manufactured from fluorescent dyes, which are dissolved beforehand in a support resin to give a solid solution, which is subsequently ground to a powder of resin particles having fluorescent properties. The preparation of such fluorescent pigments is described in EP 0 370 470, U.S. Pat. No. 2,851,424, U.S. Pat. No. 3,711,604, U.S. Pat. No. 3,856,550 and U.S. Pat. No. 2,938,878.

Fluorescent pigments particularly suitable for the present invention may thus be chosen from coloured polyamide and/or formaldehyde/benzoguanamine and/or melamine/formaldehyde/sulfonamide resins, from coloured aminotriazine/formaldehyde/sulfonamide co-condensates and/or from metallized polyester flakes and/or mixtures thereof. These fluorescent pigments may also be present in the form of aqueous dispersions of fluorescent pigments.

As fluorescent pigments particularly suitable for the present invention, mention may be made of the pink-coloured fluorescent aminotriazine/formaldehyde/sulfonamide co-condensate with a mean particle size of 3-4 microns sold under the trade name Fiesta Astral Pink FEX-1 and the blue-coloured fluorescent aminotriazine/formaldehyde/sulfonamide co-condensate with a mean particle size of 3-4.5 microns sold under the trade name Fiesta Comet Blue FTX-60 by the company Swada, or alternatively the yellow-coloured benzoguanamine/formaldehyde resin covered with formaldehyde/urea resin sold under the trade name FB-205 Yellow and the red-coloured benzoguanamine/formaldehyde resin covered with formaldehyde/urea resin sold under the trade name FB-400 Orange Red by the company UK Seung Chemical, and the orange-coloured polyamide resin sold under the trade name Flare 911 Orange 4 by the company Sterling Industrial Colors.

A second composition according to the invention may comprise a total content of fluorescent agents ranging from 0.05% to 5% by weight and preferably from 0.10% to 2.5% by weight, relative to the total weight of the second composition.

The second composition thus preferably has a dyestuff chosen from the group consisting of coloured organic pigments, or lakes, certain coloured mineral pigments (such as CI77510, CI77007), coloured hybrid pigments, coloured fluorescent agents, and a mixture thereof, preferably from the group consisting of coloured organic pigments, and coloured fluorescent agents, and a mixture thereof, preferably from the group consisting of coloured organic pigments.

These dyestuffs of the second composition may be formulated with:
at least one volatile solvent, in particular an organic solvent,
preferably at least one film-forming polymer,
preferably at least one plasticizer,
optionally, at least one dispersant, and also any other compound that is capable of preserving the optical characteristics defined previously of the reflective coat for this first composition.

Volatile Solvents

The second composition according to the invention may comprise at least one volatile solvent, which is preferably organic.

For the purposes of the invention, the term “volatile solvent” means a solvent that is capable of evaporating on contact with keratin materials in less than one hour, at room temperature and atmospheric pressure. The volatile solvent(s) of the invention are solvents that are liquid at room temperature and that have a non-zero vapour pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile solvent of the composition is preferably chosen from the group consisting of C₂-C₅ alcohols, C₅-C₁₂ alkanes, and C₃-C₆ esters and ketones, and mixtures thereof.

The term “C₂-C₅ alcohol” means an organic compound comprising at least one hydroxyl group —OH, preferably exactly one hydroxyl group —OH, and from 2 to 5 carbon atoms, and which is moreover liquid at room temperature, i.e. from 15° C. to 30° C. The C₂-C₅ alcohols that are suited to the invention especially include ethanol and isopropanol.

The term “C₅-C₁₂ alkane” means a saturated, linear or branched, cyclic or acyclic hydrocarbon-based compound, consisting exclusively of carbon and hydrogen atoms, which is moreover liquid at room temperature, i.e. from 15° C. to 30° C. In the context of the present invention, the alkanes preferably comprise from 5 to 12, preferentially from 5 to 10 and advantageously from 5 to 8 carbon atoms. The C₅-C₁₂ alkanes that are suited to the invention especially include heptane and isomers thereof, decane and isomers thereof, cyclohexane and dodecane and isomers thereof.

C₃-C₆ esters and ketones that may especially be mentioned include acetone, methyl ethyl ketone, methyl isobutyl ketone, cyclohexanone and alkyl acetates in which the alkyl group comprises from 2 to 5 carbon atoms, such as methyl acetate, ethyl acetate, propyl acetate, n-propyl acetate, isopropyl acetate, n-butyl acetate, isobutyl acetate and tert-butyl acetate.

According to a particular embodiment, the second composition comprises a total content of volatile solvent(s) of between 20% and 80% by weight, especially between 30% and 75% by weight and in particular between 40% and 70% by weight, relative to the total weight of the second composition.

Film-Forming Polymers

The second composition according to the invention may comprise at least one film-forming polymer.

For the purposes of the present invention, the term “film-forming polymer” denotes a polymer that is capable, by itself or in the presence of an auxiliary film-forming agent, of forming an isolable and especially continuous and adherent film, on a support, especially on the nails.

A single film-forming polymer or a mixture of film-forming polymers may be used in the composition.

This film-forming polymer may be chosen from the group made up of synthetic polymers, of radical type or of polycondensate type, and polymers of natural origin, and mixtures thereof.

A film-forming polymer that is suitable for use in the invention may be chosen in particular from:
cellulose-based polymers or polysaccharide derivatives, such as cellulose derivatives or guar gum derivatives. A polysaccharide derivative that is suitable for use in the invention may be a polysaccharide ester or alkyl ether.

The term “polysaccharide ester or alkyl ether” denotes a polysaccharide made up of repeat units comprising at least two identical or different rings and having a degree of substitution per saccharide unit of between 1.9 and 3, preferably between 2.2 and 2.9 and more particularly between 2.4 and 2.8. The term “substitution” denotes the functionalization of the hydroxyl groups to give ester and/or alkyl ether functions, and/or the functionalization of the carboxylic groups to give ester functions.

In other words, it may be a polysaccharide, partially or totally substituted with ester and/or alkyl ether groups. Preferably, the hydroxyl groups may be substituted with ester and/or alkyl ether functions of C₂-C₄.

Mention may in particular be made of cellulose esters, such as cellulose acetate, cellulose acetobutyrates or cellulose acetopropionates; cellulose alkyl ethers, for instance ethylcelluloses, and ethyl guars;
synthetic polymers such as homopolymers and copolymers of a compound bearing an ethylenic unit, acrylic polymers and copolymers, polyurethanes, polyesters, polyureas, silicone polymers such as silicone resins, silicone polyamides, polymers bearing a non-silicone organic backbone grafted with monomers containing a polysiloxane, polyamide polymers and copolymers, polyisoprenes, vinyl polymers, polyvinyl butyrals, alkyd resins and ketone/aldehyde resins, resins derived from aldehyde condensation products, such as arylsulfonamide-formaldehyde resins, for instance toluenesulfonamide-formaldehyde resin, epoxy/tosylamide resins, arylsulfonamide epoxy resins or ethyl tosylamide resins;
polymers of natural origin, such as plant resins, for instance dammar resins, elemi gums, copal resins, and benzoin; gums such as shellac, sandarac gum and gum mastic.

Film-forming polymers that may especially be used include the toluenesulfonamide-formaldehyde resins Ketjenflex MS80 from the company Akzo or Santolite MHP and Santolite MS 80 from the company Faconnier or Resimpol 80 from the company Pan Americana, the alkyd resin Beckosol ODE 230-70-E from the company Dainippon, the acrylic resin Acryloid B66 from the company Röhm & Haas, the polyurethane resin Trixene PR 4127 from the company Baxenden, and the acetophenone/formaldehyde resin sold under the reference Synthetic Resin SK by Degussa.

According to a particular embodiment, the film-forming polymer is chosen from polysaccharides and polysaccharide derivatives, preferably from polysaccharide ethers and esters, especially of C₂-C₄, and is more preferentially chosen from cellulose acetobutyrates, cellulose acetopropionates, cellulose acetate, ethylcelluloses, ethyl guars, nitrocellulose, epoxy/tosylamide resins and acrylic polymers, and mixtures thereof.

According to a particular embodiment, the second composition comprises a total content of film-forming polymer(s) of between 1% and 50% by weight, especially between 5% and 40% by weight and in particular between 10% and 25% by weight, relative to the total weight of the second composition.

Plasticizers

The second composition according to the invention may comprise at least one plasticizer.

For the purposes of the present invention, the term “plasticizer” denotes a compound, in more or less viscous liquid form, which, when added to a formulation of a given material, enables said material to be rendered more tacky, more adherent, more flexible, more resistant and/or easier to handle.

Plasticizers that may especially be mentioned include the usual plasticizers, such as glycols and derivatives thereof, such as diethylene glycol ethyl ether, diethylene glycol methyl ether, diethylene glycol butyl ether or else diethylene glycol hexyl ether, ethylene glycol ethyl ether, ethylene glycol methyl ether, ethylene glycol butyl ether or ethylene glycol hexyl ether; glycol esters; propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether or propylene glycol butyl ether; acid esters, especially carboxylic acid esters, such as citrates, especially triethyl citrate, tributyl citrate, triethyl acetylcitrate, tributyl acetylcitrate or 2-triethylhexyl acetylcitrate; phthalates, in particular dimethoxyethyl phthalate; phosphates, in particular tricresyl phosphate, tributyl phosphate, triphenyl phosphate or tributoxyethyl phosphate; tartrates, especially dibutyl tartrate; adipates; carbonates; sebacates; benzyl benzoate, butyl acetylricinoleate, glyceryl acetylricinoleate, butyl glyco late, camphor, glyceryl triacetate or N-ethyl-o,p-toluenesulfonamide; oxyethylenated derivatives such as oxyethylenated oils, in particular vegetable oils, such as castor oil, and more generally non-volatile oils, in particular non-volatile silicone oils, non-volatile hydrocarbon-based oils, and mixtures thereof.

Preferably, the content of plasticizer(s) is from 1% to 15% and preferably from 1% to 7% by weight, relative to the total weight of the second composition.

Needless to say, other compounds may be present in the second composition, provided that these compounds do not disrupt the translucent and coloured nature of the second composition applied.

### EXAMPLES

Several examples of first composition (base coat) and of second composition (coat covering the base coat) in accordance with the invention were prepared and compared with compositions outside the invention, especially with regard to:
contrast ratio,
luminance,
chromaticity,
   the measuring protocols of which have been given previously in this description, and evaluated with the naked eye.

Examples and Results

Several examples of preparation of first composition (known as the base coat) and of second composition (known as the top coat or covering coat) in accordance with the invention, for obtaining a depth effect of the base coat after application of one or two covering coats onto the base coat, were performed.

Examples of Nail Varnishes

Examples of first composition and counter-example

| | Composition | Composition 2 | Composition | Comparative |
|---|---|---|---|---|
| | 1 according | according | 3 according | composition |
| Ingredients with | to the | to the | to the | outside the |
| percentage content | invention | invention | invention | invention |
| Ethyl acetate | qs 100 | qs 100 | qs 100 | qs 100 |
| Nitrocellulose | 15 | 15 | 15 | 15 |
| Disteardimonium | 1 | 1 | 1 | 1 |
| hectorite (and) | | | | |
| propylene carbonate | | | | |
| (Bentone Gel ISD V | | | | |
| from Elementis) | | | | |
| Tributyl citrate | 6 | 6 | 6 | 6 |
| Alumina-titanium oxide | — | — | — | 4.3 |
| Bismuth oxychloride | 7 | 5.2 | — | — |
| Aluminium powder | — | 0 | 10.5 | — |
| TiO2 | — | 2 | — | — |
| Yellow 6 lake | — | — | — | 4 |
| Red 28 Lake | — | — | — | 4 |

Preparation Process

A coloured nail varnish is prepared by mixing, at 25° C. with suitable stirring, a film-forming fluid base (nitrocellulose) and the plasticizer (tributyl citrate) in the solvent medium (ethyl acetate); where appropriate concerning the pigments, they are optimally preground, unlike the other dyestuffs such as the nacres directly added to the base in the presence of the thixotropic agent (Bentone).

Optical Result

| | Com- | | | |
|---|---|---|---|---|
| | position 1 | Composition | Composition | Comparative |
| | according | 2 according | 3 according | composition |
| | to the | to the | to the | outside the |
| Parameters | invention | invention | invention | invention |
| Contrast ratio | 89.4 | 89.9 | 98.7 | 82.6 |
| (CR) | | | | |
| Luminance (L*) | 94.95 | 95.7 | 90.3 | 51.6 |
| Chromaticity | 2.1 | 1.7 | 0.9 | 62.5 |
| (C*) | | | | |
| Characterization | Opaque | Opaque | Opaque | Covering |
| of the base coat | lacquered | nacreous | aluminium | light red |
| deposit | white | white | | |

The first compositions 1, 2 and 3 according to the invention constitute a reflective base, unlike the comparative composition outside the invention, which is insufficiently luminous and is too coloured.

Examples of Second Composition and Counter-Example

| | | | Comparative |
|---|---|---|---|
| | Composition 1 | Composition 2 | composition |
| Ingredients with | according to the | according to the | outside the |
| percentage content | invention | invention | invention |
| Ethyl acetate | qs 100 | qs 100 | qs 100 |
| Nitrocellulose | 15 | 15 | 15 |
| Tributyl citrate | 6 | 6 | 6 |
| D&C Red 7 Lake | 0.18 | 0.05 | 1.3- |
| D&C Red 6 Lake | 0 | 0.5 | 2 |
| Red 34 Lake | — | — | 0.02 |
| TiO2 | — | — | 0.3 |

The process for preparing the second compositions is the same as that described with reference to the first compositions.

Optical Result

| | | Composition 2 | Comparative |
|---|---|---|---|
| | Composition 1 | according | composition |
| | according to the | to the | outside the |
| Parameters | invention | invention | invention |
| Contrast ratio (CR) | 28.6 | 32.2 | 76.8 |
| Luminance (L*) | 56.1 | 53.6 | 39.2 |
| Chromaticity (C*) | 62 | 69 | 53.8 |
| Characterization of the | Translucent | Translucent red | Opaque red |
| base coat deposit | fuchsia | | |
| (covering coat) | | | |

The second compositions 1 and 2 according to the invention constitute a translucent and coloured covering, unlike the comparative composition outside the invention, which is opaque.

Other examples of second compositions in accordance with the invention were prepared and allowed the Applicant to place the optimum optical result limits:

| Ingredients with | Composition | Composition | Composition |
|---|---|---|---|
| percentage | 3 according to | 4 according to | 5 according to |
| content | the invention | the invention | the invention |
| Ethyl acetate | qs 100 | qs 100 | qs 100 |
| Nitrocellulose | 15 | 15 | 15 |
| Tributyl citrate | 6 | 6 | 6 |
| D&C Red 6 lake | — | — | 0.18 |
| Ferric | 0.002 | 0.02 | — |
| ammonium | | | |
| ferrocyanide | | | |
| D&C Yellow 5 | 0.22 | — | — |
| Lake | | | |
| Red 34 Lake | — | 0.11 | — |

Optical Result

| | | | Composition 4 | |
|---|---|---|---|---|
| | Composition | | according to | |
| | 3 according | Composition 4 | the invention | Composition 5 |
| | to the | according to the | (spreading 2 × | according to the |
| Parameters | invention | invention | 300 μm) | invention |
| Contrast ratio (CR) | 23 | 37 | 67.9 | 25.4 |
| Luminance (L*) | 87.7 | 47.3 | 36.3 | 63.4 |
| Chromaticity (C*) | 48.1 | 51.8 | 43.2 | 60.9 |
| Characterization of | Translucent | Translucent | Translucent | Translucent |
| the covering coat | green-yellow | violet | violet | orange |
| | | (spreading | (spreading to a | |
| | | according to the | thickness of | |
| | | protocol of the | 600 μm) | |
| | | description at | | |
| | | 300 μm) | | |

Result of the Successive Application of the Base Coat to the Nail Followed by the Covering Coat onto the Base Coat

When a coat of first composition according to the invention, or base coat, such as the first composition 1, is applied to a nail, and a coat of second composition, or covering coat, such as the second composition 2, is then applied onto the base coat, the translucent and coloured nature of the covering coat applied onto this reflective base coat makes it possible to observe a surprising and unexpected depth effect of said base coat. Generally, only one base coat is applied, but one or two covering coats may be applied onto said base coat so as to vary this depth effect.

In contrast, by applying a first comparative composition outside the invention and then a second composition according to the invention, such a depth effect is not obtained. Similarly, by applying a first composition 1 according to the invention and then a second comparative composition outside the invention, such a depth effect is not obtained.

To illustrate now a modification of the lightness of the colour of the assembly obtained while at the same time allowing an intense colour to be obtained, a base coat according to the invention of first composition 2 according to the invention was applied, followed, respectively, by one and two covering coats of second composition 1 according to the invention, and a reduction in luminance may then be observed while at the same time maintaining high chromaticity.

Optical Result

| | First composition 2 | |
|---|---|---|
| | according to the invention | First composition 2 |
| | covered with a second | according to the |
| | composition 1 according | invention covered |
| | to the invention | with a second |
| | (spreading 300 μm | composition 1 |
| | according to the protocol | according to the |
| | defined in the patent | invention (spreading |
| Parameters | application) | 600 μm) |
| Contrast ratio (CR) | 94.9 | 93.5 |
| Luminance (L*) | 56.2 | 47.6 |
| Chromaticity (C*) | 64.9 | 64.3 |
| Characterization of | Intense fuchsia | Deep intense fuchsia: |
| the covering coat | | the shade is less light, |
| deposit | | but still maintains |
| | | strong chromaticity |

Nail varnishes are not the only fields of application of the present invention: this invention is thus transposable to other technical fields, such as lip products, such as lipsticks, or eye products, such as liners.

Examples of Lip Products

Examples of First Composition

Example of preparation of a white base for the preparation of a first composition in accordance with the invention:

| | | White |
|---|---|---|
| Phases | Ingredients | base |
| A | Isododecane | 19.41 |
| | TRIMETHYL SILOXYSILICATE (SR | 18.86 |
| | 1000 from MOMENTIVE | |
| | PERFORMANCE MATERIALS) | |
| | DISTEARDIMONIUM HECTORITE (and) | 27.73 |
| | PROPYLENE CARBONATE (BENTONE | |
| | GEL ISD V from ELEMENTIS) | |
| B | Isododecane | 19.22 |
| | NYLON-611/DIMETHICONE | 12.57 |
| | COPOLYMER (DOW CORNING 2-8179 | |
| | GELLANT) | |
| | C30-45 ALKYLDIMETHYLSILYL | 0.55 |
| | POLYPROPYLSILSESQUIOXANE (and) | |
| | PARAFFIN (DOW CORNING SW-8005 | |
| | C30 RESIN WAX) | |

### Preparation Process

mix the starting materials of phase A at room temperature
in a separate mixer, mix the starting materials of phase B and heat to 75° C., then cool to room temperature
mix the starting materials of phase B with phase A.

To accentuate the contrast ratio of this white base, several examples of reflective metal particles, optionally in combination with pigments, were added to make a first covering composition according to the invention:

| Ingredients with | Composition | Composition | Composition | Composition | Composition |
|---|---|---|---|---|---|
| percentage | according to | according to | according to | according to | according to |
| content | invention 1 | invention 2 | invention 3 | invention 4 | invention 5 |
| D&C Yellow 6 | 0.09 | 0.1 | — | — | 0.072 |
| Lake | | | | | |
| Red 28 | 0.08 | 0.16 | 0.88 | — | — |
| D&C Red 22 | — | — | — | 0.18 | 0.13 |
| Lake | | | | | |
| Titanium dioxide | 2.4 | 2.6 | — | — | — |
| Alumina (and) | 9.14 | 11.93 | 28.32 | 15.82 | 15.87 |
| titanium dioxide | | | | | |
| Prepared white | qs 100 | qs 100 | qs 100 | qs 100 | qs 100 |
| base | | | | | |

Preparation Process:

The nacres are first added to the white base beforehand. Next, the pigments are ground in a three-roll mill and added to this nacreous white base.

### Optical Results:

| | Composition | Composition | Composition | Composition | Composition |
|---|---|---|---|---|---|
| | according to | according to | according to | according to | according to |
| Parameters | invention 1 | invention 2 | invention 3 | invention 4 | invention 5 |
| Contrast ratio | 95.1 | 96.5 | 96.9 | 95.4 | 95.5 |
| (CR) | | | | | |
| Luminance | 88.9 | 84.8 | 82.8 | 89.9 | 88.6 |
| (L*) | | | | | |
| Chromaticity | 13 | 23.8 | 31.8 | 16.2 | 15.5 |
| (C*) | | | | | |
| Characterization | Opaque | Opaque | Opaque | Opaque | Opaque |
| of the | nacreous | nacreous | nacreous pink | nacreous | nacreous |
| base coat | pink pastel | pink pastel | pastel | peach pastel | peach pastel |
| deposit | | | | | |

Second Composition

A second composition in accordance with the invention was prepared as follows:

| Ingredients with percentage | Composition according |
|---|---|
| content | to the invention |
| Red 28 Lake | 2.46 |
| Ozokerite | 6.27 |
| Beeswax | 2.64 |
| Bis-diglyceryl polyacyladipate-2 | 24.47 |
| (Softisan 649 from Cremer | |
| Oleo) | |
| Trimethylpentaphenyltrisiloxane | qs 100 |
| (Dow Corning PH-1555 HRI | |
| Cosmetic Fluid) | |

The pigments were preground and the ingredients were mixed at 80° C., and the mixture then cast in stick form.

Optical Results

| | Composition according to the |
|---|---|
| Parameters | invention |
| Contrast ratio | 49.5 |
| (CR) | |
| Luminance (L*) | 48.4 |
| Chromaticity (C*) | 59.9 |
| Characterization of | Intense fuchsia |
| the covering coat | |
| deposit | |

When a first composition according to the invention was applied to the lips, and then, once the first composition was dry, a second composition was applied onto said first composition, a depth effect was observed.

Needless to say, these examples are not limiting: other examples using other starting materials may be used for these examples of varnishes and lipsticks. Similarly, other applications may be imagined, such as a mascara application.

Examples of Eye Products (Liners, Mascara)

A first composition for forming a base coat and a second composition for forming a coat covering the base coat may be formulated as follows, for illustrative purposes.

First Composition

| | FIRST |
|---|---|
| | COMPOSITION |
| | ACCORDING TO |
| | THE |
| INGREDIENT WITH PERCENTAGE CONTENT | INVENTION |
| Bismuth oxychloride | 13 |
| STYRENE/ACRYLATES/AMMONIUM | 40 |
| METHACRYLATE COPOLYMER (and) SODIUM | |
| LAURETH SULFATE (and) CAPRYLYL | |
| GLYCOL (SYNTRAN 5760 CG from | |
| INTERPOLYMER) | |
| Hydroxypropylcellulose | 1 |
| Preserving agent | qs |
| Water | qs 100 |

This first composition may be prepared via a conventional preparation process, but it will preferably be advisable not to grind the bismuth oxychloride.

Second Composition

| | SECOND |
|---|---|
| | COMPOSITION |
| | ACCORDING TO |
| INGREDIENT WITH | THE |
| PERCENTAGE CONTENT | INVENTION |
| Blue 1 Lake | 2.5 |
| Acrylic acid/isobutyl | 40 |
| acrylate/isobornyl acrylate | |
| copolymer (Mexomer PAS from | |
| Chimex) | |
| Preserving agent | qs |
| Isododecane | qs 100 |

This second composition may be prepared via a conventional preparation process, but it will preferably be advisable to grind the organic pigment used with a three-roll mill.

It is understood that, in the context of the present invention, the weight percentages given for a compound or a family of compounds are always expressed as weight of solids of the compound in question.

Throughout the application, the term “comprises one” or “includes one” should be understood as meaning “comprising at least one” or “including at least one”, unless otherwise specified.

1. An assembly for making up a keratin material, the assembly comprising:
a first composition capable of forming a reflective coat; and
a second composition being a translucent and colored composition.

2. The assembly of claim 1, wherein the first composition comprises at least one reflective metal particle.

3. The assembly of claim 2, wherein the at least one reflective metal particle is selected from the group consisting of a pure metal particle, a composite metal particle, a multilayer interference pigment, and a mixture thereof.

4. The assembly of claim 2, wherein the a least one reflective metal particle is present in a total content ranging from 2% to 40% by weight, relative to a total weight of the first composition.

5. The assembly of claim 1, wherein the first composition is capable of forming a color selected from the group consisting of white, pink, metallic, and pastel.

6. The assembly of claim 1, wherein the first composition has a contrast ratio CR of greater than or equal to 75%, a chromaticity C* of less than or equal to 40, and optionally a luminance L* of greater than or equal to 60.

7. The assembly of claim 1, wherein one of the first composition comprises at least one volatile solvent.

8. The assembly of claim 1, wherein the first composition comprises at least one film-forming polymer present in a total content ranging from 1% to 50% by weight relative to a total weight of the first composition.

9. The assembly of claim 1, wherein the first composition comprises at least one plasticizer.

10. The assembly of claim 1, wherein the second composition comprises at least one dyestuff selected from the group consisting of a colored organic pigment, a colored lake, a colored mineral pigment, a colored hybrid pigment, a colored florescent agent and a mixture thereof.

11. The assembly of claim 1, wherein the second composition comprises at least one dyestuff, present in a total content ranging from 0.05% to 5% by weight relative to a total weight of the second composition.

12. The assembly of claim 1, wherein the second composition has a contrast ratio CR ranging from 15% to 70%, a chromaticity C* ranging from 30 to 90, and optionally a luminance L* of greater than or equal to 40.

13. The assembly of claim 1, wherein the second composition comprises at least one volatile solvent.

14. The assembly of claim 1, wherein the second composition comprises at least one film-forming polymer present in a total content ranging from 1% to 50% by weight relative to a total weight of the second composition.

15. The assembly of claim 1, wherein the second composition comprises at least one plasticizer, present in a total content ranging from 1% to 15% relative to a total weight of the second composition.

16. A process for making up a keratin material, the process comprising applying the assembly of claim 1 to the keratin material.

17. The assembly of claim 1, which is adapted to function as an assembly for making up keratin materials selected from the group consisting of nails, lips, eyelids, eyelashes, eyebrows and a contour of the eyes.

18. The process of claim 16, comprising:
applying the first composition to the keratin material to form a reflective coat; and then
applying the second composition onto the first composition.
